(19) United States
(12) Patent Application Publication
(10) Pub. No.: US 2007/0224286 A1
(43) Pub. Date: Sep. 27, 2007

(54) Process for Preparing Calcium Phosphate Self-Setting Bone Cement, the Cement So Prepared and Uses Thereof

(76) Inventors: Thundyil Raman Narayanan Kutty, Bangalore (IN); Devarasu Thirunavukkarasu, Bangalore (IN)

Correspondence Address: DEWITT ROSS & STEVENS S.C., 8000 EXCELSIOR DR, SUITE 401, MADISON, WI, 53717-1914, US

(21) Appl. No.: 11/569,531

(22) PCT Filed: Jun. 24, 2004
(86) PCT No.: PCT/IN04/00183

§ 371 Date: Nov. 22, 2006

(51) Int. Cl. A61K 33/42; C08K 3/32

(52) U.S. Cl. 424/602; 523/116

(57) ABSTRACT

A calcium phosphate self-setting cement is invented by using diffusion controlled solid liquid heterogeneous reaction between tetracalcium phosphate (Ca₄(PO₄)₂O, TTCP) fine powder and solution of di-potassium hydrogen phosphate (K₂HPO₄). Fine powders of tetracalcium phosphate is introduced into the di-potassium hydrogen phosphate solution and homogenized well to form a cement paste. The cement paste was allowed to set at room temperature (25±5° C.). In the cement paste calcium is leached from the tetracalcium phosphate (TTCP) fine particles as Ca(OH)₂ into the aqueous phase. Leaching of calcium continues until the Ca/P ratio changes from 2 to 1.67, which corresponds to hydroxyapatite. Calcium hydroxide thus formed reacts with the phosphate ions (p0₄³⁻) that exist in the liquid phase, and the reaction leads to in-situ precipitation of hydroxyapatite as the reaction product, which leads to interparticle entanglement in the cement paste, thereby forming self-setting bone cement.

### CROSS-REFERENCE TO RELATED APPLICATIONS

Priority is hereby claimed to co-pending International Application Serial No. PCT/IN2004/000183, filed 25 Jun. 2004, which was published as WO 2006/001028, on 5 Jan. 2006.

This invention relates to a process of preparing self-setting cement using water solouble di-potassium hydrogen phosphate as reactants and a natural biopolymer as pore forming medium, the cement so prepared and uses thereof.

### BACKGROUND

The first material proposed for bone repair in early 19^{th} century was plaster of Paris, which converts eventually to gypsum. By 1965 it was clear that this material was resorbed faster than the over growth of new bone, and therefore was been abandoned. Several ceramics have been proposed since then. Bioglass and Ceravital belong to a group of surface-active ceramics that form strong bond with bone mediated by large apatite crystals [1]. In a similar way, apatite- or wollastonite-containing glass ceramics have also been developed [2] which bonds strongly to bones, and are also mediated by apatite layers [3]. Further developments are still going on [4]. The interface bond strength between bioactive glasses and bone is still increasing after one year of implantation in an acetabular dog bone [5]. These materials are not biodegradable. Glass-ionomer cements which are widely used as dental restorative material have also been proposed as bone cements [6]. Xenobiotic components such as aluminium are leached from such materials and may accumulate in the soft tissues; whether this type of material is also bioactive is still questionable. Recently bioactive cement has been developed which is based on the system CaO—SiO₂—P₂O₅—CaF₂, a glass powder in combination with an aqueous solution of ammonium phosphate [7]. These materials are not biodegradable either. Some biodegradable formulations have been proposed for repair of bone defects. A chitosan sol was used as carrier for a powder containing a mixture of hydroxyapatite, zinc oxide and calcium oxide [8]. Its biological behavior has not yet been reported. A composite of poly (l-lactide) and hydroxyapatite has been studied in a transcortical implantation model in goats [9]; up to three months the interface bonding increased. However, later the bonding diminished due to the dominant effect of poly(l-lactide) resorption without sufficient bone on growth. Several other materials for bone repair and bone substitution have been proposed. However up to now none of them has proved to be a major value in surgery. One of the serious drawbacks is that most of the materials cannot be formulated into the desired form during operation. In this respect it is too early to judge the practical value of bioglass cements [8]. A revolution in orthopedic surgery was about to take place in the early 1960s, when Sir John Charnley presented the preliminary results of new methods for the fixation of prosthesis to bone and bone defects [10]. The idea was making a cement consisting of self-curing PMMA with a filler material. The fillers are hydroxyapatite [11], bioactive glass cement powder [12]. However the disadvantages are apparent as reported in [13], evolution of large amount of heat during polymerization, setting shrinkage after polymerization, and cardiac arrest due to monomer toxicity as explained in the literature [14]. In summery the behavior of PMMA bone cement indicates its lack of biocompatibility. The first step in overcoming the above mentioned limitations was made in 1983 by the introduction of cement formulation consisting of calcium phosphates [15]. The cement formulation contain powder mixtures of tetracalcium phosphate (TTCP, Ca₄(PO₄)₂O) and dicalcium phosphate anhydride (DCPA, CaHPO₄) or dicalcium phosphate dihydrate (DCPD, CaHPO₄.2H₂O). These cements can be made in molded forms during operation or simply injected into the bone defects. Since then, self-hardening calcium phosphate cements (CPCs) based on mixtures of several calcium phosphates are proposed for maxillofacial and bone defect repair [16] in no- or low-load bearing orthopedic applications. These cements are formed interaoperative and are hardening in-vivo to micro porous and non-ceramic hydroxyapatite of low crystallinity.

Despite the large number of formulations, CPCs can only have three different end phases, namely hydroxyapatite, brushite and amorphous calcium phosphate [17]. Hence the CPCs are classified into two categories: (i) apatite CPCs and (ii) brushite CPCs. Most of the research efforts have been put in towards apatite CPCs, despite some interesting features of brushite CPCs. The cements are based on acid-base reactions between calcium orthophosphate combinations and cement formation is based on pH dependent solubilities of calcium phosphates. CPCs are made of two or more calcium phosphate powders with an aqueous solution as reaction medium. Upon mixing, the calcium phosphate(s) dissolves and precipitates into a less soluble calcium phosphate. During the precipitation reaction, the calcium phosphate crystals grow and became entangled, thus providing mechanical rigidity to the cement [17]. Reaction mechanisms proposed in the cement formation from different calcium phosphates are classified into two groups based on pH dependent solubilities [18]: (i) Cements formed at pH≦4 which are obtained using acidic calcium phosphates (MCPM, Ca(HPO₄)₂) or phosphoric acid with α/β-tricalcium phosphate (α/β-TCP, (Ca₃(PO₄)₂) for brushite (DCPD, CaHPO₄.2H₂O) [19] formation and (ii) pH above 4.2 hydroxyapatite (HAP, Ca₅(PO₄)₃ (OH)) is formed, the reaction can occur according to an acid-base reaction i.e relatively acidic calcium phosphate (CaP) phase reacting with a basic CaP to produce nearly neutral and less soluble CaP. Reactants for HAP cements are crystalline calcium phosphates such as TTCP [20] and α/β-tricalcium phosphate α/β-TCP, (Ca₃(PO₄)₂) [20] combining with slightly acidic compounds such as DCPD or DCPA. Because of different rates of dissolution of reactants, a setting reaction will only occur if the kinetic solubilities of all the components are congruent, hence this normally adjusted via particle size/specific surface area of the reactant particles [21-22]. Other than the above mentioned reactants, use of calcium hydroxide (Ca(OH)₂) along with DCPD or DCPA or TCP as reactants to form CPCs containing HAP was also attempted [23]. In-vitro studies on calcium phosphate cements (CPCs) show that they are osteoconductive, i.e after implantation in bone defects they are rapidly integrated into the bone structure, after which they are transformed into new bone by cellular activities of osteoblasts and these osteoblasts take care of the local bone remodeling. Over calcium phosphate ceramics which must be preshaped, the CPCs have the advantage that they can be molded during operation. This means that these materials adapt immediately to the bone cavity and so obtain good osteointegration.

In addition to the calcium phosphate cements (CPCs), dense or porous calcium phosphate ceramics especially hydroxyapatite are often applied on strong and load-bearing core materials for biological fixation or osteointegration of load bearing implants such as hip steps and dental roots. Porous calcium phosphate ceramics are also expected to play important roles in treating bone problems with emerging tissue engineering approach, as it involves loading proper cells into porous ceramics (scaffolds) and implanting the cell-loaded scaffold into the host body for achieving bone tissue regeneration. The term ‘tissue engineering’ encompasses a variety of approaches to the same goal and as such a bone tissue engineering approach has been defined as combination of one or more of the following osteoconductive material, osteoprogemtor cells and osteoconductive growth factor.

Experimental studies on the implantation of porous calcium phosphate ceramics showed that the degree of infiltration of living tissues into the pores and formation of new bone depended greatly on the pore characteristics such as porosity, pore size, pore size distribution and pore shape. It was claimed [24] that a minimum pore size of 100 μm is necessary for the porous implant materials to function well and pore size greater than 200 μm is an essential requirement for osteoconduction. In addition to the application of porous calcium phosphate ceramics in tissue engineering, use of the same biomaterials to deliver biologically active agents is an attractive concept because local administration of certain therapeutic agents is often the most effective method of treatment. Since biomaterials are generally used to reconstruct or replace tissues and joints they are usually placed in a wound healing environment in the body. In the majority of cases the most favorable biological response is rapid tissue repair. Since we are aware that human growth hormone (hGH) is a potent stimulator of bone repair. Incorporation of growth hormone in porous hydroxyapatite for local delivery in the site of the wound is yet another attractive concept in controlled delivery system. The development of controllable, long-term and effective release systems for delivery of growth hormones and other growth factors may improve the wound healing and tissue repair.

Apart from the sustained and effective release of biologically active agents, delivery of drugs from porous hydroxyapatite has also been proposed in the recent past to replace the other drug carriers those are mentioned below. Drug carriers of different types have been developed since 1971 using biodegradable [25] and non-biodegradable polymers. The former includes poly(lactic acid) (PLA) [25], poly(glycolic acid) (PGA) [26], poly ε-caprolactone (Pε-CL), their co-polymers [26], poly(acrylic acid) [27], poly(acrylic acid)+chitosan mixtures and chitosan [28] a polysaccharide, and non-biodegradable polymer porous polyurethane [29] for the controlled delivery of drugs. However the drug carriers based on PLA, PGA and their copolymers are well accepted as drug delivery systems because of their good biocompatibility and novel drug release behavior. The byproducts of the PLA, PGA and their copolymers is carbon-dioxide, and water molecules, those are drained out as metabolic waste from the human body. However these nanoparticles are not ideal carriers for hydrophilic drugs such as peptides, proteins and some anticancer drugs because of their hydrophobic properties. To improve their hydrophilic properties different types of nanoparticle have been developed as hydrophilic drug carriers, such as poly(ethyleneglycol) (PEG)-modified polyester nanoparticles are the promising carriers for the hydrophilic drugs due to the hydrophilic property and other outstanding physico-chemical and biological properties of PEG [30]. Preparation and evaluation of environmental sensitive, self regulated drug carriers were also attempted in the recent past [31]. However the hydrophobic/hydrophilic nanoparticles have limitations, such as their preparation procedures involve multiple steps, requirement of the use of organic solvents and surfactants as well as sonication or homogenization and storage of the drug loaded carriers in specified conditions leads to expensive methods of drug administration.

In the above mentioned drug carriers based on biodegradable and non-biodegradable polymers the drug loaded carriers are prepared by water-in-oil direct emulsion or inverse emulsion techniques [32] and spray drying [33] processes. In these techniques particle size control is a serious drawback as the particle size varies from 200 nm to 10 μm, with wide particle size distribution. Hence it poses difficulties during the administration of the carriers, for example in vein injection the particle size should be ≦2 μm [33]. To overcome the above mentioned limitations, porous hydroxyapatite scaffolds have been proposed as carriers of drugs [34] and biologically active agents [35] for controlled and sustained delivery. Biocompatibility, bone bonding and bone regeneration properties are the advantages of the hydroxyapatite ceramics to use them as potential candidates for controlled release applications.

Different methods for the preparation of useful porous scaffolds have been reported in literature. A number of papers have reported the methods for the preparation of useful porous scaffolds. The earliest study could be the fabrication of porous HAP by duplicating the macro porous structure of natural ocean corals [36]. Polyvinyl butyral (PVB) particles were used [37] as pore formers to prepare porosity controlled HAP ceramics through both solid process as well as the liquid process. Open cell hydroxyapatite foams were produced through the technique of gel-casting [38]. Porous hydroxyapatite ceramics were also produced by impregnating polyurethane foams with slurry containing HAP powder, water and additives [39]. Fabrication of porous hydroxyapatite is also attempted [40] by coating the calcium phosphate cement on polyurethane foam, then firing the cement at higher temperature. Recent report [41] explains the method of preparation of porous hydroxyapatite scaffolds by combining gel-casting and polymeric sponge methods. The difficulties involved in the usage of polyurethane as the template for the fabrication of porous hydroxyapatite are (i) tendency of the slurry to drain almost completely from parts of the foam due to interfacial wetting behavior of the foam with the slurry, (ii) in reticulated foam the slurry tend to accumulate at the points between rods of polymer rather than along the length of the rods themselves, (iii) multiple time soaking of the foam in the prepared slurry to ensure that the template is completely filled with slurry and (iv) evolution of copious fumes in the firing stages.

### OBJECTS OF THE PRESENT INVENTION

The main objective of the present invention is to establish a simplified processing procedure for the formulation of self-setting cement and fabrication of porous calcium phosphate for tissue engineering, controlled release of drugs and biologically active agents. In developing such a process, the present invention is focused on finding suitable reactants for the preparation of self-setting cements. It is the primary objective to find suitable pore forming medium, which can be used along with the cement to prepare calcium phosphate without posing any problem during or after processing to the working personnel and the environment as well. Yet another objective of the invention is to develop a processing procedure for the preparation of porous calcium phosphate of the desired characteristics such as porosity, pore size, pore size distribution, and pore connectivity which can be used as scaffold, as a carrier for tissue engineering and as a carrier for the controlled release of drugs and biologically active agents.

This invention thus provides a process for preparing calcium phosphate self-setting cement using cassava/tapioca pearls as pore forming medium in the self-setting cement matrix. The pearls are dispersed in distilled water in the solid to liquid ratio of 1.2 to 1.3 ml/gm. The dispersed pearls absorb water and swell correspondingly at room temperature (25±5° C.). Water absorbed pearls are added to the freshly prepared cement paste and homogenized well to ensure their uniform distribution in the cement matrix. The mixture is allowed to set at room temperature (25±5° C.). The set specimens are dried free of residual water at temperatures ranging from 50 to 120° C. Dried specimens are fired at higher temperature varied from 950 to 1250° C. to obtain porous hydroxyapatite with open as well as closed cell-structure. Porous hydroxyapatite specimens are imbibed with Vitamin C (a nutrient) and tetracycline (an antibiotic) in suitable solvent media which are then dried under controlled conditions. The imbibed specimens release the nutrients or antibiotics when placed in living body.

In the accompanying drawings;

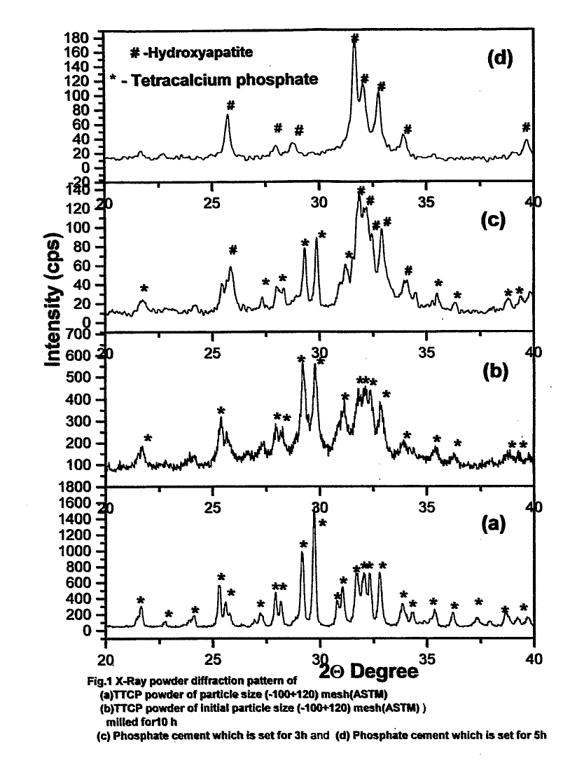

FIG. 1 illustrates the X-Ray powder diffraction pattern of
(a) TTCP powder of particle size (−100+120) mesh (ASTM);
(b) TTCP powder of initial particle size (−100+120) mesh (ASTM) milled for 10 h;
(c) Phosphate cement which is set for 3 h;
(d) Phosphate cement which is set for 5 h;

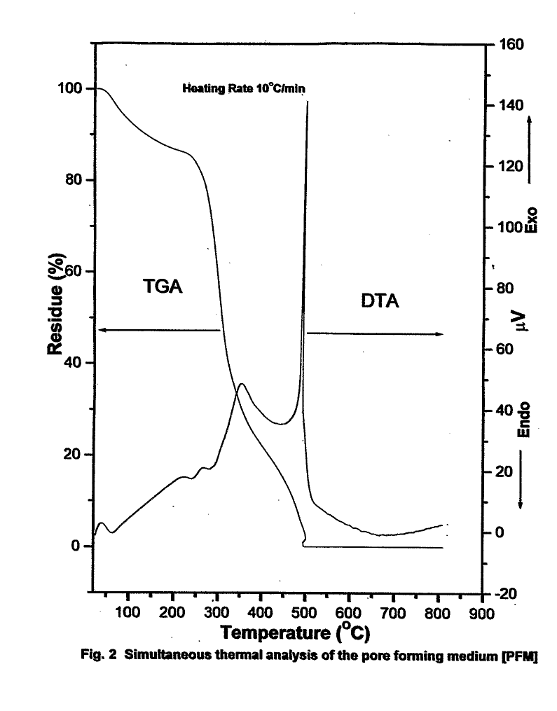

FIG. 2 illustrates the simultaneous thermal analysis of the pore-forming medium [PFM];

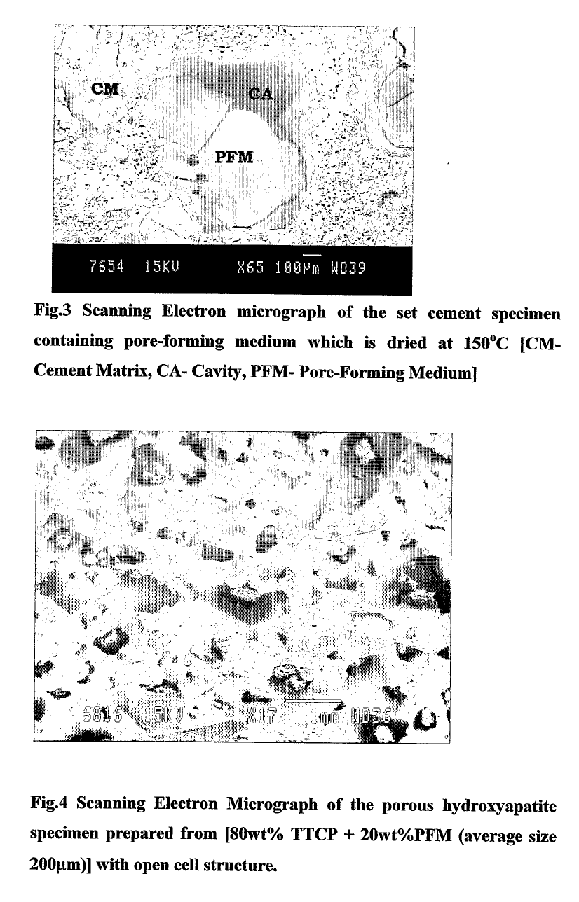

FIG. 3 illustrates tge scanning Electron micrograph of the set cement specimen containing pore-forming medium which is dried at 150° C. [CM-Cement Matrix, CA-Cavity, PFM-Pore-Forming Medium];

FIG. 4 illustrates the scanning Electron Micrograph of the porous hydroxyapatite specimen prepared from [80 wt % TTCP+20 wt % PFM (average size 200 μm)] with open cell structure; and

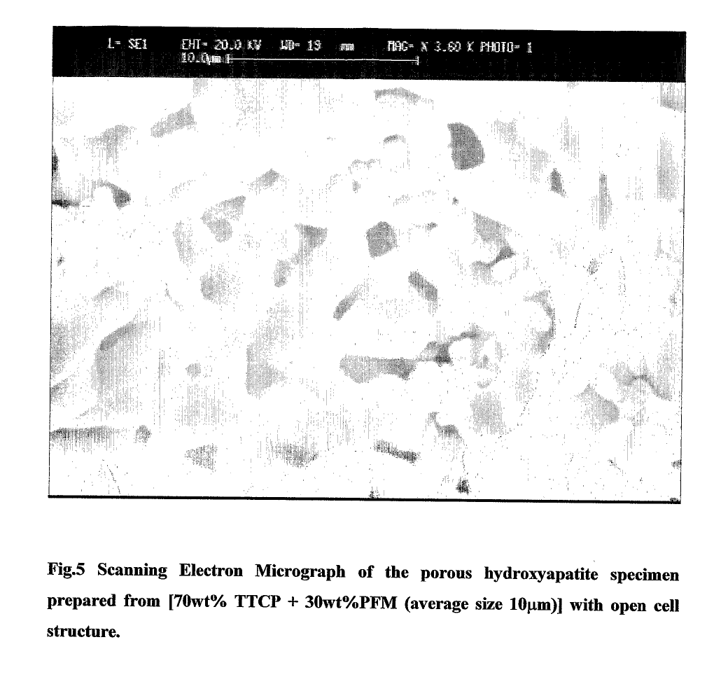

FIG. 5 illustrates the scanning Electron Micrograph of the porous hydroxyapatite specimen prepared from [70 wt % TTCP+30 wt % PFM (average size 10 μm)] with open cell structure.

### MATERIALS USED AS REACTANTS FOR THE PREPARATION OF SELF-SETTING CEMENTS

The basic principle for the preference to use specific materials as reactants is that it should be biocompatible. The reaction between the reactants should lead to setting into cement at human body temperature. The reactants should not produce or leave any harmful byproducts during or after cementation reaction. The phase content of the set cement should be one of the calcium phosphates as it is biocompatible. The reactants should be easily available or can be synthesized in the laboratory conditions. The reactants which meet all the above mentioned requirements are water soluble di-potassium hydrogen phosphate (K₂HPO₄) and tetracalcium phosphate (Ca₄(PO₄)₂O, TTCP) fine powder. Di-potassium hydrogen phosphate is highly soluble in water at room temperature. Tetracalcium phosphate is synthesized by the high temperature solid state reaction from a mixture of calcium pyro phosphate (Ca₂P₂O₇) and calcium carbonate (CaCO₃) fine powders. The reaction can be written as

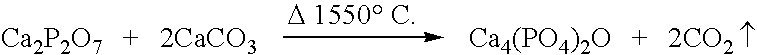

FIG. 1(a) shows the X-ray powder diffraction pattern of the TTCP powder obtained from the above reaction. TTCP thus obtained was ground to fine powders by milling in planetary ball mill with a liquid medium such as acetone or toluene. All the chemicals used were of analytical grade purity from well known manufacturers.

General Method and Procedure Used for the Preparation of Self-Setting Cement:

One of the reactants in the cement formation, tetracalcium phosphate (TTCP) fine powder was prepared by milling of coarser particle (−100+120 mesh ASTM) in a planetary ball mill for different durations varied from 5 h to 20 h in presence of a liquid medium such as acetone followed by drying off the solvent. The other reactant in the cement formation was taken in the form of solution by dissolving di-potassium hydrogen phosphate in distilled water. The reactants, fine powder of tetracalcium phosphate was added in to the di-potassium hydrogen phosphate solution and homogenized well to form a cement paste. In the reactant mixture solid to liquid ratio is varied from 0.45 to 0.55 ml/gm. The paste is transferred into a container made of plastic or glass and the container is closed well to avoid the escape of moisture from the cement paste.

In the cement paste calcium is leached from the tetracalcium phosphate (TTCP) fine particles as Ca²⁺ ions into the aqueous phase. Leaching of calcium continues until the Ca/P ratio changes from 2 to 1.67 which corresponds to hydroxyapatite. Leached Ca²⁺ ions form calcium hydroxide (Ca(OH)₂) by reacting with water molecules from the aqueous phase. Calcium hydroxide thus formed reacts with the phosphate ions (PO₄³⁻) which exist in the liquid phase, and the reaction leads to in-situ precipitation of hydroxyapatite as the product. The in-situ precipitated hydroxyapatite leads to interparticle binding in the cement paste, thereby forming self-setting bone cement at room temperature (25±5° C.). The above explained reactions can be depicted by the following chemical equations;

Reaction.1: Formation of Calcium hydroxide in the cement paste.
3 Ca₄(PO₄)₂O+3H₂O→Ca₁₀(PO₄)₆ (OH)₂+2(Ca(OH)₂) (1)

Reaction.2: Precipitation of hydroxyapatite in the cement paste.
5(Ca(OH)₂)+3K₂HPO₄→Ca₅(PO₄)₃(OH)+6KOH (2)

Based on this reaction, every one mole of TTCP is reacting with 0.4 mole of phosphate (PO₄³⁻) to form hydroxyapatite by decreasing the Ca/P ratio from 2 to 1.667. Porosity of the set cement is measured by the pycnometric method [42]. The crystalline phase content of the set cement was determined by X-ray powder diffraction method. Cold crushing strength of the cement specimens were measured by uniaxial loading using an INSTRON-8032 universal testing machine. Procedure for preparation and evaluation of the specimens was followed by the standard method available in literature [43]

### EXAMPLE

150 gm of tetracalcium phosphate powder of initial particle size −100+120 mesh (ASTM) was milled for 10 h in a planetary ball mill with acetone as the liquid medium in an agate container. Milled powder was dried free of liquid medium. FIG. 1 (b) shows the X-ray diffraction pattern of the tetracalcium phosphate fine powder obtained after milling in a planetary mill for 10 h. Di-potassium hydrogen phosphate solution of 2.185M concentration was prepared by dissolving 380.59 gm of the salt in 1000 ml of distilled water at room temperature (25±5° C.). Dried tetracalcium phosphate powder was added to 65 ml of 2.185M di-potassium hydrogen phosphate solution. The reactants were homogenized well by stirring with glass or plastic rod or with a mechanical stirrer to form a cement paste. The cement pastes were transferred into plastic or glass containers and were kept closed to retain the moisture content all through the cementing reaction at room temperature (25±5° C.). After the cementation reaction set specimens were removed from the containers and dried free of water. Porosity of the dried specimens was measured by pycnometric method and was found to be 45% by volume. Phase content of the set cement was identified by X-ray powder diffraction method. The XRD pattern [FIG. 1 (c)] shows the presence of the TTCP and hydroxyapatite phases, which indicates the incompletion of the reaction in the cement which is allowed to set for 3 h. Whereas the phase content [FIG. 1 (d)] in the cement that is allowed set for 5 h is end member hydroxyapatite. Cold crushing strength of the cylindrical cement specimens of dimensions 6 mm (diameter)×12 mm (height) was measured by uniaxial loading using INSTRON-8032 a universal testing machine. Cement specimens were sintered at different temperatures varied from 1100° C. to 1250° C. for 3 h. Sintered specimens with 97% theoretical density were obtained after sintering at 1250° C. for 3 h. Crushing strength of the cement specimens varied from 50 to 75 MPa.

Material used as Pore-Forming Medium [PFM] for porous Calcium Phosphate Fabrication from Self-Setting Cement:

The basic principle for the preference to use a particular material as pore forming medium is that it should be stable in the processing conditions, such as with limited solubility in water or other solvents such as ethanol that are commonly used in the processing steps. Since the bone cement formation involves in-situ precipitation reactions, the pore forming medium should not interfere or affect the precipitation reaction process. Pore-forming medium should not form any stable reaction products with the reactants during or after processing. PFM should not be hazardous during or after the processing conditions. It is important that they should not pose any problem for the environment. It is desirable that the PFM should be readily available or synthesized in simple ways and economically viable. PFM should be compatible for any cementing reactions. Based on these essential requirements, the material optimized in the present invention as pore forming medium is starch pearls derived from tubers of cassava or tapioca plants. Preparation of starch pearls is reported elsewhere [44] and these pearls are readily available in the market as food stuff. Plant source from which these granules are prepared is given below.

| | |
|---|---|
| Botanical Name: | Manihot Esculanta |
| Class: | Dicotylendonae |
| Sub Class: | Monochlamydea |
| Order: | Unisexuals |
| Family: | Euphorbiaceae |

These starch pearls are used as foodstuff all over the world, particularly in tropical countries.

Properties and Known Applications of the Cassava/Tapioca Pearls:

The starch pearls can be crushed into particles of different sizes varying from millimeters to micrometers. Crushing of the cassava pearls become easier after cooling in dry ice or liquid nitrogen (Cryogrinding). Pearls absorb water in minutes when they are introduced into water and swell correspondingly. When the pearls are allowed to dry, they shrink back to near original size as they loose water by the dehydration process. However, the solid mass, which is prepared from dehydration of the starch solution, does not show the swelling during the water re-absorption. Swelling is also not observed when the starch is re-precipitated by adding ethanol. These observations clearly indicate that the swelling property is related to globular rather than linear conformation of the polymer as also the intermolecular interactions and the nature of packing of the polymeric carbohydrate molecules. Quantitative analysis of the starch pearls shows that it is composed of 88% carbohydrate with 0.5% protein and minute amounts of fat and traces of B vitamins the remaining being moisture. The alkali content are fairly low namely 0.0242% Na₂O and 0.001% K₂O. Simultaneous thermal analysis [TGA/DTA] of the starch pearls is given in FIG. 2 Weight loss at 50° C. is due to the removal of absorbed moisture. The broad exotherm from 150 to 225° C. is due to the partial decomposition of the pearls and associated weight loss is around 12%. The exothermic peak from 300 to 450° C. indicates pyrolysis and the accompanying oxidation of the gaseous products in air. The corresponding weight loss in TG is 78%. The sharp exotherm at 500° C. indicates the removal of residual carbon formed from the decomposition of the pearls leaving no residue at 550° C. These pearls are commonly used to prepare soups, cakes and puddings. In cookery, it is mainly used as sauce thickener. In industry, it is used as textile stiffener. In the fine powder form, it can be used as a gelling agent.

Materials used for Preparation of Porous Calcium Phosphate from Self-Setting Cement:

| Materials | Grade | Source |
|---|---|---|
| Reactants | | |
| Tetracalcium phosphate | Analytical reagent | Prepared by high |
| fine powder (Ca₄(PO₄)₂O) | | temperature (1550° C.) |
| | | solid state reaction |
| | | between |
| | | Ca₂P₂O₇ and CaCO₃ |
| | | taken in 1:2 mol ratio. |
| Di-potassium hydrogen | Analytical reagent, | Prepared by dissolving |
| phosphate solution | Water soluble | K₂HPO₄ (E-MERCK) |
| | | salt in distilled water. |
| Pore-Forming Medium | | |
| Starch Pearls | Food grade | Local sources |

General Method and Procedure for the Preparation of Porous Hydroxyapatite from Self-Setting Cement:

Fine powder of tetracalcium phosphate was stirred into the di-potassium hydrogen phosphate solution of 2.185M and homogenized well by stirring with a glass rod or with a mechanical stirrer to prepare cement paste of 200 to 250 gm batches. Solid to liquid ratio in the cement paste varied from 0.45 to 0.55 ml/gm. The pore forming medium, cassava or tapioca pearls was dispersed in water, where the solid to liquid ratio is 1.2 to 1.3 ml/gm. The water+pearls mixture was added to the cement forming paste and homogenized by string with a glass rod or with a mechanical stirrer. In the mixture, tetracalcium phosphate powder content was varied from 70 to 80 wt % and the pore forming medium from 20 to 30 wt % on dry basis. The mixture of cement paste and pore-forming medium was transferred to containers made of plastic or glass and the containers were kept closed to retain the moisture in the mixture all through the cementing reaction. The setting reaction was carried out at room temperature. After the cementing reaction the set specimens were removed from the containers and dried at higher temperatures ranging from 50° C. to 120° C. to ensure that the specimens were dried-up well to nearly free of water. While drying the specimens, the pore-forming medium releases water and shrinks back to the original size. This is accompanied by detachment from the surrounding medium of cement matrix thereby generating cavities (or pores) in the cement matrix. It is evident from the SEM (Scanning Electron Microscopy) picture [FIG. 3] of the specimens which are dried at 150° C. wherein the PFM and the cement matrix are indicated as PFM and CM respectively. Cavities generated are clearly discernible from the cement matrix and the pore forming medium. Dried specimens were taken to higher temperatures for sintering. The schedule of sintering for the specimens was guided by the data from simultaneous thermal analysis [TGA/DTA] of the dry specimens. Accordingly, the specimens were heated at constant heating rate of 150° C./h up to 175° C. and the specimens were kept at this temperature whereby they show partial decomposition. Then the temperature was further raised to 225° C. at constant heating rate of 150° C./h at which pyrolysis of the PFM took place, as was evident from the TGA. This was associated with the broad exothermic peak in DTA. At a constant heating rate 180° C./h the temperature was then increased to 450° C. at which CO₂+CO evolution takes place. Temperature was then raised to 550° C. at 50° C./h whereby the residual carbon was eliminated by oxidation. The specimens were then taken to the sintering temperatures varied from 1100° C. to 1250° C. for 3 h duration, while the heating rate was maintained at 240° C./h. The specimens were cooled to room temperature at the rate of 480° C./h.

Porosity of the sintered specimens was measured using the pycnometric method. Pore size and shape of the ceramics were determined using the SEM micrographs by the intercept method. To determine the mechanical behavior of the sintered porous ceramics, cold crushing strengths were measured under uni-axial loading at constant strain rate.

Porous hydroxyapatite specimens were loaded with Vitamins such as ascorbic acid and antibiotics such as tetracycline by using suitable solvents at room temperature (25±5° C.). Loading of the drugs varied from 2 to 5% by weight of the specimens on dry basis. Controlled release of the drugs were studied by immersing the drug loaded specimens in the simulated body fluid (SBF) which is maintained at a pH of 7.5 at room temperature (25±5° C.). It was found that the porous hydroxyapatite specimens of 2 cm³ with 5 wt % loading releases drugs in a controlled way for a minimum of two weeks. Hence the porous hydroxyapatite specimens are suitable candidates for controlled and sustained delivery of drugs.

### EXAMPLES

(i) Tetracalcium phosphate 80 wt %+Cassava Pearls 20 wt %

120 gm of cassava pearls of average size 200 μm was dispersed in 14 ml of distilled water by stirring with glass rod. 300 gm of tetracalcium phosphate was added in 130 ml of 2.185M di-potassium hydrogen phosphate solution and were homogenized well by stirring with glass rod or with mechanical stirrer to prepare the cement paste. Cassava+water mixture was added to the cement paste and stirred well to ensure the homogeneous distribution of the pearls in the cement paste. Then the cement paste+cassava pearls mixture transferred to a container made of plastic or glass and kept closed well to ensure that the moisture content in the mixture is the same all through the cementing reaction. The setting reaction is carried out for 5 h at room temperature. Then the set specimens were dried in air oven at higher temperatures from 50° C. to 120° C. to remove the residual water. After drying, the specimens were taken to higher temperature for sintering. Sintering was carried out in a pre-determined schedule, which was guided by the data from simultaneous thermal analysis [TGA/DTA] [FIG. 4] of the dry specimens. Accordingly, the specimens were heated at constant heating rate 150° C./h up to 175° C. and the specimens were kept at this temperature whereby they show partial decomposition. The temperature was further raised to 225° C. at constant heating rate of 150° C./h at which, pyrolysis of the PFM is seen in TGA. This is associated with the broad exothermic peak in DTA. At a constant heating rate 180° C./h, the temperature was then increased to 450° C. at which CO₂+CO evolution takes place. Temperature was then raised to 550° C. at 50° C./h whereby the residual carbon was eliminated by oxidation. The specimens were then sintered at 1250° C. for 3 h, while the heating rate was maintained at 240° C./h. The specimens were cooled to room temperature at the rate of 480° C./h. Porosity of the sintered specimens was measured using the pycnometric method and is found to be 65% by volume. SEM picture of a sintered specimen is shown. FIG. 5 indicating the open cell structure of the porous hydroxyapatite. Pore size varies from 150 to 300 μm. Cold crushing strength of the sintered porous hydroxyapatite was found to be 290 kPa under uniaxial loading at constant strain rate.

(ii) Tetracalcium phosphate 70 wt %+Cassava Pearls 30 wt %

150 gm of cassava pearls of average size of 10 μm was dispersed in 120 ml of distilled water by stirring with glass rod. 350 gm of tetracalcium phosphate was added to 160 ml of 2.185M di-potassium hydrogen phosphate solution and were homogenized well by stirring with glass rod to prepare the cement paste. Cassava+water mixture was added to the cement paste and stirred well to ensure the homogeneous distribution of the pearls in the cement paste. The cement paste+cassava pearls mixture was transferred to a container made of plastic or glass and kept closed well to ensure that the moisture content in the mixture is retained all through the cementing reaction. The setting reaction is carried out for 5 h at room temperature. Then the set specimens were dried in air oven at higher temperatures from 50 to 120° C. to remove the residual water. After drying, the specimens were taken to higher temperature for sintering. Sintering was carried out in a predetermined schedule, which in turn was guided by the data from simultaneous thermal analysis [TGA/DTA] of the dry specimens. Accordingly, the specimens were heated at constant heating rate of 150° C./h up to 175° C. and the specimens were kept at this temperature whereby they show partial decomposition. Then the temperature was further raised to 225° C. at constant heating rate of 150° C./h at which, pyrolysis of the PFM is seen in TGA. This is associated with the broad exothermic peak in DTA. At a constant heating rate of 180° C./h the temperature was then increased to 450° C. at which CO₂+CO evolution takes place. Temperature was then raised to 550° C. at 50° C./h whereby the residual carbon was eliminated by oxidation. The specimens were then sintered at 1250° C. for 3 h, while the heating rate was maintained at 240° C./h. The specimens were cooled to room temperature at the rate of 480° C./h.

Porosity of the sintered specimens was measured using pycnometric method and is found to be 65% by volume. SEM picture of the sintered specimen is shown FIG. 6, which has the open cell structure of the porous hydroxyapatite. Pore size varies from 5 to 10 μm. Cold crushing strength of the sintered porous hydroxyapatite was found to be 290 kPa under uniaxial loading at constant strain rate.

Advantages of the Present Invention:

1. The pore forming medium does not interfere or affect the in-situ precipitation reaction of calcium phosphate and hence the cassava/tapioca pearls do not hinder the cement formation.
2. The dried cement specimens can be handled easily as the struts are strong enough to withstand the changes in pressure during handling.
3. The cavities generated in the set cent specimens remain undisturbed while the cassava/tapioca pearls are burned off in the sintering stages.
4. The burning of the cassava/tapioca pearls does not pose any problem to the environment as they do not produce any copious toxic fumes during sintering of the set cement specimens.

### REFERENCES

1. L. L. Hench. R. J. Splinter, T. K. Greenlee, and W. C. Allen, Bonding mechanisms at the interface of ceramic prosthetic material, J. Biomed. Mat. Res. Symp. 5(1971) 117-141.
2. T. Kokobo, S. Ito, M. Shigamatsu, S. Sakka. and T. Yamamuro, Mechanical properties of a new type of apatite-containing glass-ceramics for prosthetic application, J. Mater. Sci., 20 (1985) 2001-2004.
3. T. Nakamura, T. Yamamuro, S. Higashi, T. Kokubo and S. Ito, A new glass-ceramic for bone replacement: evaluation of its bonding to bone tissue, J. Biomed. Mater. Res, 19 (1985) 685-698.
4. L. A. Wolfe and Boyde, Biocompatibility tests on a novel glass-ceramic system, J. Appl. Biomaterials, 3 (1992) 217-224.
5. S. Yoshii, T. Nakamura, T. Yamamuro, M. Oka, H. Takai and S. Kotani, Glass-ceramic implant in acetabular bone defect: an experimental study, J. Appl. Biomaterials, 3 (1992) 245-249.
6. I. M. Brook, G. T. Craig and D. J. Lamp, In vitro interaction between primary, bone organ cultures, glass-ionomer cements and hydroxyapatite/tricalcium phosphate ceramics, Biomaterials 12 (1991) 179-186.
7. Y. Taguchi, T. Nakamura, T. Yamamuro, N. Nishimura, T. Kokubo, E. Takahata and S. Yoshihara, A bioactive glass powder-ammonium hydrogen phosphate composite for repairing bone defects, J. Appl. Biomaterials, 1 (1990)217-223.
8. M. Ito, In vivo properties of a chitosan-bonded hydroxyapatite bone-filling paste, Biomaterials, 12 (1991) 41-45.
9. C. C. P. M. Verheyen, J. R. de Wijn, C. A. van Blitterswyk, K. deGroot and P. M. Rozing, Hydroxyapatite/poly(l-lactide) composites: an animal onpush-out strengths and interface histology, J. Biomed. Mater. Res., 27 (1993) 433-444.
10. Charnely, J. Bonding of prosthesis to bone by cement, J. Bone Joint Surg. 46B (1964) 518.
11. J. Dandurand, V Delpech, A. Lebugle, A Lamure and C. Lacabanne, Study of the mineral-organic linkage in an apatitic reinforced bone cement, J. Biomed. Mater. Res., 24 (1990) 1377-1384.
12. W. Hennig, B. A. Blemke, H. Bromer, K. K. Deutscher, A. Gross and W. Age, Investigations with bio activated PMMs, J. Biomed. Mater. Res., 13 (1979) 89-99.
13. R. S. M. L, Complicaciones de las artoplasttias totales de cadera, Salvate Editores, Barcelona, 1987.
14. A. F. Newens, and R. G. Volz, Severe hypotension during prosthetic hip surgery with acrylic bone cement, Anesthesiology, 36 (1972) 298-300.
15. W. E. Brown and L. C. Chow, A new calcium phosphate setting cement, J. Dental Research, 62 (1983) 672.
16. Driessens, F. Planell, J. Gil F. calcium phosphate bone cements, In: Wise, D L. et al Encylopedic hand book of biomaterials and bio engineering, Part B, vol 2, New York: Marcel Dekker, 1995.
17. M. Bohner, Calcium phosphates in medicine: from ceramics to calcium phosphate cements, Injury. Int. J. Care Injured, 31(2000) S-D 37-47.
18. Driessens, F. Planell, J. Ginbera, M. Gil, F. E. Fernandez, Best, S. M. Calcium phosphate bone cements for clinical applications, Part 1: Solution chemistry, J. Mat. Sci: Mater In Med, 10 (1999) 169-176.
19. Mirtchi A A et. al Calcium phosphate cements: action of setting regulators on the properties of the beta-tricalcium phosphate-monocalcium phosphate system, Biomaterials 10 [7] 1989 475-480.
20. L. C. Chow, Development of self-setting calcium phosphate cements, J. Ceram. Soc. Jpn. 99 (1991) 954-964.
21. Driessens, F. Planell, J. Boltong, E. Fernandez, and Ginbera, M P. Influence of particle size of the powder phase in the setting and hardening behavior of a calcium phosphate cement. In: Sedel, L et al Bioceramics Vol. 10 New York Elsievier Science 1997 481-484.
22. Otsuka, M. Matsuda Y. Suwa Y. Fox J L. Higuchi Wis., Effect of particle size of metastable calcium phosphates on mechanical strength of a novel self-setting bioactive calcium phosphate cement, J. Bio Med Mat. Res 29 (1995) 25-32.
23. S. Takagi, L. C. Chow and K. Ishikawa, Formation of hydroxyapatite in new calcium phosphate cements, Biomaterials 19(1998) 1593-1599.
24. J. Biomed Mat. Res Symp. 2 [1] (1970) 269
25. Sinclair, R. G., Slow release pesticide system: Polymers of lactic and glycolic acids as ecologically beneficial, cost effective encapsulating materials, Environ. Sci. Technol. 7 (1973) 955-956.
26. Ryu. J G. et al, Clonazepam release from core shell type nanoparticles of Poly(ε-Caprolactone)/PEG/Poly(ε-Caprolactone) triblock co-polymers. Int J. Pharm 200[2] (2000) 231-242
27. J. S. Ahn et al Release of triamcinolone acetonide from mucoadhesive polymer composed of chitosan and poly(acrylic acid) in vitro, Biomaterials, 23 (2002) 1411-1416.
28. Yang Hu et al, Synthesis and characterization of chitosan-poly(acrylic acid) nanoparticles, Biomaterials, 23 (2002) 3193-3201.
29. K. L. Shantha and K. Pnaduranga Rao, Drug release behavior of polyurethane microspheres, Journal of applied polymer Science, 50 (1993) 1863-1870.
30. Heald, C R. Et al, Self consistent field modeling of poly(lactic acid)-poly(ethylene glycol)particles, Colloids Surf. A: Physico Chem Engg Aspects 179[1] (2001): 79-91.
31. Yong Qiu and Kinam Park, Environment-sensitive hydro gels for drug delivery, Advanced Drug Delivery Reviews 53 (2001) 321-339.
32. Jalil, R. and J. R. Nixon, Biodegradable PLA and poly(LA-co-GA) microcapsules: Problems associated with preparative techniques and release properties, J. Microencapsultaion, 7 (1990) 297-325.
33. He. P. Davis S S, Illum, L. Chitosan microspheres prepared by spray drying, Int J. Pharm 187 (1999) 53-65.
34. D. J. A. Netz, P. Sepulveda, V. C. Pandolfelli, A. C. C. Spadaro, J. B. Alencastre, M. V. L. B. Bentley and J. M. Marchetti., Potential use of gel-casting hydroxyapatite porous ceramic as an implantable drug delivery system. International journal of pharmaceutics 213 (2001) 117-125.
35. D. M. Arm, A. F. Tencer, S. D. Bain and D. Celino, Effect of controlled release of platelet-derived growth factor from porous hydroxyapatite implant on bone in growth, Biomaterials 17(1996) 703-709.
36. D. M. Roy, S. K. Linnehan, Nature 247 (1974) 220.
37. D-M. Liu, Preparation and characterization of porous hyrdroxy apatite bio ceramics via a slip-casting route Ceram. Int. 24 (1998) 441.
38. P. Sepulveda, F. S. Ortega, M. D. M. Innocentini, and V. C. Pandolfelli, Properties of highly porous hydroxyapatite obtained by gel-casting of foams, J. Am. Ceram. Soc, 83(12)(2000) 3021.
39. J. Tian, J. Mater. Sic. 36 (12) (2001) 1543.
40. X. Miao, Y. Hu, J. Liu, and A. P. Wong. “Porous calcium phosphate ceramics prepared by coating polyurethane foams with calcium phosphate cements”. Materials Letters 58 (2004) 397-402.
41. H. R. Ramay and M. Zhang, Preparation of porous hydroxyapatite scaffolds by combination of the gel-casting and polymer sponge methods, Biomaterials 24 (2003) 3293-3302.
42. Annual Book of ASTM Standards, 1995 Section 15, Vol 15.01 Designation C133-94 pp 29-33.
43. Annual Book of ASTM Standards, 1995 Section 15 Vol. 15.01 pp 115-116.
44. Lilia. S. Collado, and Harold Corke, “Pasting Properties of Commercial and Experimental Starch Pearls,” Carbohydrate Polymers 1998 35 89-96.

1. A process for preparing calcium phosphate self-setting bone cement using water soluble di-potassium hydrogen phosphate, tetracalcium phosphate as reactants and a natural biopolymer as pore forming medium by,
i. dissolving the di-potassium hydrogen phosphate in distilled water, followed by loading with tetracalcium phosphate fine powder and setting the cement at room temperature (25±5° C.),
ii. adding cassava/tapioca pearls as pore forming medium in the cement for the fabrication of porous hydroxyapatite wherein the swelled pearls shrink back to near original size on drying to produce cavities in the green specimen to generate porosity,
iii. drying the set cement at different temperatures varying from 50 to 120° C.; and
iv. firing the set cement in a predetermined schedule at higher temperatures ranging from 950 to 1250° C. to yield either porous or dense calcium phosphate ceramics.

2. A process as claimed in claim 1, wherein the di-potassium hydrogen phosphate is dissolvable in water at room temperature to which tetracalcium phosphate powder is loaded and homogenized to form a cement paste.

3. A process as claimed in claim 2, wherein calcium is leached out from the tetracalcium phosphate fine particles into the liquid phase as Ca²⁺ ions to form calcium hydroxide (Ca(OH)₂) with water molecules in the liquid medium.

4. A process as claimed in claim 3, wherein the leaching continues until the Ca/P ratio changes from 2 to 1.67 at which stage hydroxyapatite is formed.

5. A process as claimed in claim 3, wherein the calcium hydroxide (Ca(OH)₂) reacts with phosphate ions (PO₄³⁻) that exists in the liquid phase, thereby precipitating hydroxyapatite in-situ.

6. A process as claimed in claim 5, wherein the precipitated hydroxyapatite leads to interlocking of particles to form the cement at room temperature (25±5° C.).

7. A process as claimed in claim 1, wherein diffusion controlled solid-liquid heterogeneous reaction is achieved with tetracalcium phosphate fine particles and di-potassium hydrogen phosphate solution, and the cement formation is completed within 5 to 10 minutes.

8. A process as claimed in claim 1, wherein the porous hydroxyapatite with open as well as closed structure is prepared by using cassava/tapioca pearls as pore forming medium in the self-setting bone cement matrix.

9. A process as claimed in claim 7, wherein the cement paste is prepared by mixing the di-potassium hydrogen phosphate solution with tetracalcium phosphate powder in the solid to liquid ratio 0.45 to 0.55 ml/gm.

10. A process as claimed in claim 7, wherein the cassava/tapioca pearls are dispersed in water in the solid to liquid ratio of 1.2 to 1.3 ml/gm.

11. A process as claimed in claim 9, wherein the cement paste and the cassava/tapioca pearls+water mixture were homogenized to achieve uniform distribution of pore forming medium in the cement paste matrix.

12. A process as claimed in claim 11, wherein the set cement is dried at different temperatures varying from 50° C. to 120° C. for the complete removal of residual water from the specimens.

13. A process as claimed in claim 11, wherein cassava/tapioca pearls used as pore forming medium for the fabrication of porous hydroxyapatite is crushed under cryogenic conditions to obtain different sieve size of 1 to 500 mesh (ASTM)

14. A process as claimed in claim 9, wherein during dispersion of the cassava/tapioca pearls in water the pearls absorb water and swell in size correspondingly in the suspension.

15. A process as claimed in claim 11, wherein drying of the set cement is accompanied by the loss of water from the cassava/tapioca pearls and shrink back to near original size thereby producing cavities or pores in the green cement matrix by interfacial detachment.

16. A process as claimed in claim 15, wherein the dried cement specimens, were sintered at higher temperature varied from 950 to 1250° C. to obtain porous cellular ceramics with open as well as closed cell structure and relatively high in strength.

17. A process as claimed in claim 16, wherein the set cement obtained retains the structural integrity even after firing at higher temperatures.

18. A self-setting cement composition comprising di-potassium hydrogen phosphate tetracalcium phosphate in the range of 70 to 80% by weight and 20 to 30% by weight of a natural biopolymer as pore forming medium, the biopolymer is selected from cassava/tapioca pearls.

19. A self-setting carrier for the controlled release of drugs and biologically active agents comprising the self cement composition prepared by the process as claimed in claim 1.
